(19) United States
(12) Patent Application Publication
(10) Pub. No.: US 2023/0128875 A1
(43) Pub. Date: Apr. 27, 2023

(54) METHODS AND APPARATUS FOR VIEWING CONTRAST-ENHANCED ULTRASOUND IMAGES AND DYNAMIC IMAGES

(71) Applicant: SHENZHEN MINDRAY BIO-MEDICAL ELECTRONICS CO., LTD., Shenzhen (CN)

(72) Inventors: Maodong SANG, SHENZHEN (CN); Zebing WANG, SHENZHEN (CN); Lei ZHU, SHENZHEN (CN)

(21) Appl. No.: 17/971,507

(22) Filed: Oct. 21, 2022

(30) Foreign Application Priority Data
Oct. 26, 2021 (CN) 202111246924.6

(51) Int. Cl. A61B 8/08; A61B 8/00

(52) U.S. Cl. CPC A61B 8/481; A61B 8/46; A61B 8/5207

(57) ABSTRACT

Disclosed are methods and apparatus for viewing a contrast-enhanced ultrasound image and a dynamic data. The method comprises: receiving a first operation, for setting a first viewing range by a first browsing step length that is multiple frames; in response to the first operation, positioning the image data to a viewing neighborhood containing the first viewing range; receiving a second operation, on the view neighborhood by a second browsing step length that is a single frame; in response to the second operation, determining a current image frame corresponding to when the second operation is performed in the viewing neighborhood, and further positioning the image data to an adjacent frame of the current image frame to the user to view frame by frame. As such, doctors are helped to accurately locate desired image frames to significantly improve browsing efficiency with convenient operation and high user-friendliness to save time and reduce workload.

### TECHNICAL FIELD

The present disclosure relates to medical imaging apparatus, methods, and media, and more particularly, to methods and apparatus for viewing contrast-enhanced ultrasound (CEUS) images and dynamic images.

### BACKGROUND OF THE INVENTION

Ultrasound, which can be generated and detected simply and inexpensively, can be penetrated deep into human tissues without causing tissue damage, making it ideal for non-invasive biomedical imaging. When encountering a scatterer, ultrasound may be scattered, and the scattering intensity thereof is related to the size and shape of the scatterer, as well as the difference of acoustic impedance between the scatterer and the surrounding tissue. The scattering of blood to ultrasound is very weak, so an “anechoic” symbol may be appeared on ordinary ultrasonic instruments in case of blood scattering ultrasound. If a medium (microbubbles) with different acoustic impedance from blood is added to the blood, the scattering in the blood will be enhanced, which is the basic principle of contrast-enhanced ultrasonography. CEUS for tissues utilizes this principle; specifically, ultrasound contrast agent, that is, a solution containing microbubbles, is injected into the body, and the contrast agent enters organs and tissues to develop or enhance the imaging of the organs and tissues, thereby providing an important basis for clinical diagnosis.

In recent years, CEUS imaging has played an increasingly important role in the differential diagnosis and ablation assessment of cardiovascular, liver, thyroid, and breast diseases. Taking liver tumors as an example, compared with normal tissues, the micro-blood flow in malignant tumors is often more abundant, and the typical manifestation of its CEUS images is that the microbubbles in the focus region enter and subside faster than those in normal tissues. Generally speaking, in order to distinguish this hemodynamic difference between normal tissues and malignant tissues, CEUS is required to have a certain imaging frame rate. The commonly used 2D real-time CEUS imaging frame rate is usually set to 10-15 fps at present. The recently introduced high frame rate imaging technology can increase the imaging frame rate to dozens or even hundreds of frames per second.

Because the survival time of the ultrasound contrast agent in the body is limited, doctors often stores all contrast-enhanced image data during this period of time during contrast examination. Data about the focus region can be reviewed and browsed repeatedly to improve the confidence of diagnosis. On the other hand, such data can be used for clinical research, academic lectures or clinical teaching. However, when browsing or replaying, there is such a problem that: when doctors start to browse the contrast-enhanced data frame by frame, it often takes a long time to find effective information frames because of the large number of contrast-enhanced data frames with high frame rate.

### SUMMARY OF THE INVENTION

Therefore, there is a need for a method and apparatus for viewing CEUS images and dynamic images, which can help doctors to significantly improve the browsing efficiency of dynamic image data with high frame rates and accurately locate a single image frame or a small range of image frames that the doctor expects to view. Moreover, similar to the interactive operation adopted in traditional browsing, the interactive operation performed by doctors is convenient and high user friendly, saving the doctor's time and notably reduce the doctor's workload.

According to a first aspect of the present disclosure, there is provided a method for viewing a CEUS image. The data of the contrast-enhanced ultrasound image is generated under an imaging mode by a contrast-enhanced ultrasound apparatus. The imaging mode comprises a first imaging mode and a second imaging mode, an imaging velocity under the first imaging mode is faster than that under the second imaging mode, a range of a first browsing step length corresponding to the imaging mode is determined upon the imaging velocity corresponding to the imaging mode, the range of the first browsing step length corresponding to the imaging mode is positively correlated with the imaging velocity corresponding to the imaging mode. The method comprises: by means of a processor, when the contrast-enhanced ultrasound image data to be viewed is opened for manual viewing: receiving a first operation, by a user via an interactive unit, for setting a first viewing range by the first browsing step length that is multiple frames interactive unit; positioning the contrast-enhanced ultrasound image data to a viewing neighborhood containing the first viewing range in response to the first operation; receiving a second operation performed, by the user via the interactive unit, on the viewing neighborhood containing the first viewing range by a second browsing step length that is a single frame; and in response to the second operation, determining a current image frame corresponding to when the second operation is performed by the user in the viewing neighborhood containing the first viewing range, and further positioning the contrast-enhanced ultrasound image data to an adjacent frame of the current image frame for the user to view frame by frame.

According to a second aspect of the present disclosure, there is provided a method for viewing a CEUS image. The data of the contrast-enhanced ultrasound image is generated under an imaging mode by a contrast-enhanced ultrasound apparatus. The imaging mode comprises a first imaging mode and a second imaging mode, an imaging velocity under the first imaging mode is faster than that under the second imaging mode, the imaging velocity is positively correlated with a first browsing step length. The method may comprise: by means of a processor, when the contrast-enhanced ultrasound image data to be viewed is opened for manual viewing: receiving a first operation, by the user via an interactive unit, for setting a first viewing range by the first browsing step length that is multiple frames; positioning the contrast-enhanced ultrasound image data to a viewing neighborhood containing the first viewing range in response to the first operation; receiving a fifth operation by the user for setting an automatic viewing mode; and in response to the fifth operation, enabling the contrast-enhanced ultrasound image data to begin to play automatically in a single-frame manner in the viewing neighborhood containing the first viewing range, the playback speed of playing automatically being lower than the imaging velocity of the second imaging mode.

According to a third aspect of the present disclosure, there is provided a method for viewing a dynamic image for viewing dynamic image data comprising multiple image frames. The method for viewing may comprise: by means of a processor, when the dynamic image data comprising multiple image frames to be viewed is opened for manual viewing: receiving a first operation, by a user via an interactive unit, for setting a first viewing range by a first browsing step length that is multiple frames; in response to the first operation, positioning the dynamic image data to a viewing neighborhood containing the first viewing range; receiving a second operation performed, by the user via the interactive unit, on the viewing neighborhood containing the first viewing range by a second browsing step length, the second browsing step length being smaller than the first browsing step length; and in response to the second operation, determining a current image frame corresponding to when the second operation is performed by the user in the viewing neighborhood containing the first viewing range, and further positioning the dynamic image data to an image frame that is the current image frame moving forward or backward by one single second browsing step length for the user to view frame by frame.

According to a fourth aspect of the present disclosure, there is provided an apparatus for viewing a CEUS image, wherein the data of the CEUS image is generated under an imaging mode by a CEUS apparatus. The imaging mode comprises a first imaging mode and a second imaging mode, an imaging velocity under the first imaging mode is faster than that under the second imaging mode, the imaging velocity is positively correlated with a first browsing step length. The apparatus for viewing comprises: an interactive unit configured to perform a first operation by the user for setting a first viewing range by the first browsing step length that is multiple frames, and perform a second operation by the user on a viewing neighborhood containing the first viewing range by a second browsing step length that is a single frame; a processor configured to perform the method for viewing a CEUS image according to each embodiment of the present disclosure; and a display configured to show an image or interface corresponding to the positioned viewing range under the control of the processor.

According to a fifth aspect of the present disclosure, there is provided an apparatus for viewing a CEUS image, wherein the data of the CEUS image is generated under an imaging mode by a CEUS apparatus. The imaging mode comprises a first imaging mode and a second imaging mode, an imaging velocity under the first imaging mode is faster than that under the second imaging mode, the imaging velocity is positively correlated with a first browsing step length, and the apparatus for viewing comprises: an interactive unit, a processor and a display. The interactive unit may be configured to perform a first operation by the user for setting a first viewing range by the first browsing step length that is multiple frames, and perform a fifth operation by the user for setting an automatic viewing mode. The processor may be configured to receive a first operation, by the user via an interactive unit, for setting a first viewing range by the first browsing step length that is multiple frames; position the CEUS image data to a viewing neighborhood containing the first viewing range in response to the first operation; receive a fifth operation by the user for setting an automatic viewing mode; and in response to the fifth operation, enable the CEUS image data to begin to play automatically in a single-frame manner in the viewing neighborhood contain the first viewing range, the playback speed of playing automatically being lower than the imaging velocity of the second imaging mode. The display may be configured to show an image corresponding to the positioned viewing range under the control of the processor.

According to a sixth aspect of the present disclosure, there is provided an apparatus for viewing a dynamic image. The apparatus for viewing may include an interactive unit, a processor and a display. The interactive unit may be configured to perform a first operation by a user for setting a first viewing range by a first browsing step length that is multiple frames, and perform a second operation by the user on a viewing neighborhood containing the first viewing range by a second browsing step length, the second browsing step length being smaller than the first browsing step length. The processor may be configured to, when dynamic image data including multiple image frames to be viewed is opened for manual viewing, detect a first operation performed by the user; in response to the first operation, position the dynamic image data to a viewing neighborhood containing the first viewing range; detect a second operation performed by the user; and in response to the second operation, determining a current image frame corresponding to when the second operation is performed by the user in the viewing neighborhood containing the first viewing range, and further positioning the dynamic image data to an image frame that is the current image frame moving forward or backward by one single second browsing step length for the user to view frame by frame. The display may be configured to show an image corresponding to the positioned viewing range.

By means of the methods and apparatus for viewing CEUS images and dynamic images according to each embodiment of the present disclosure, doctors can be helped to significantly improve the browsing efficiency of dynamic image data with high frame rates and be accurately located a single image frame or a small range of image frames that the doctor expects to view. Moreover, similar to the interactive operation adopted in traditional browsing, the interactive operation performed by doctors is convenient and high user friendly, saving the doctor's time and notably reduce the doctor's workload.

### BRIEF DESCRIPTION OF THE DRAWINGS

The features, advantages, and technical and industrial implications of exemplary embodiments of the present invention will be described below with reference to the accompanying drawings, wherein like reference numerals refer to like elements, and wherein:

FIG. 1 is a schematic configuration of a CEUS imaging apparatus according to an embodiment of the present disclosure;

FIG. 2 is an exemplary flowchart of a CEUS examination according to an embodiment of the present disclosure;

FIG. 3 is a schematic flowchart of Example 1 of a method for viewing a CEUS image according to an embodiment of the present disclosure;

FIG. 4 is a schematic flowchart of Example 2 of a method for viewing a CEUS image according to an embodiment of the present disclosure;

FIG. 5 is a schematic interface for manually viewing and browsing CEUS images according to an embodiment of the present disclosure;

FIG. 6 is a schematic interface for automatically viewing and browsing CEUS images according to an embodiment of the present disclosure;

FIG. 7 is a schematic flowchart of Example 3 of a method for viewing a CEUS image according to an embodiment of the present disclosure;

FIG. 8 is a schematic flowchart of Example 4 of a method for viewing a dynamic image according to an embodiment of the present disclosure;

FIG. 9 is a schematic flowchart of Example 5 of a method for viewing a CEUS image according to an embodiment of the present disclosure;

FIG. 10 is a schematic interface for manually viewing and browsing CEUS images according to an embodiment of the present disclosure; and

FIG. 11 is a schematic interface for setting a first viewing range according to an embodiment of the present disclosure.

### DETAILED DESCRIPTION

Hereinafter, embodiments of the present invention will be described; however, the present invention is not intended to be limited to the embodiments. All components of the embodiments are not always essential.

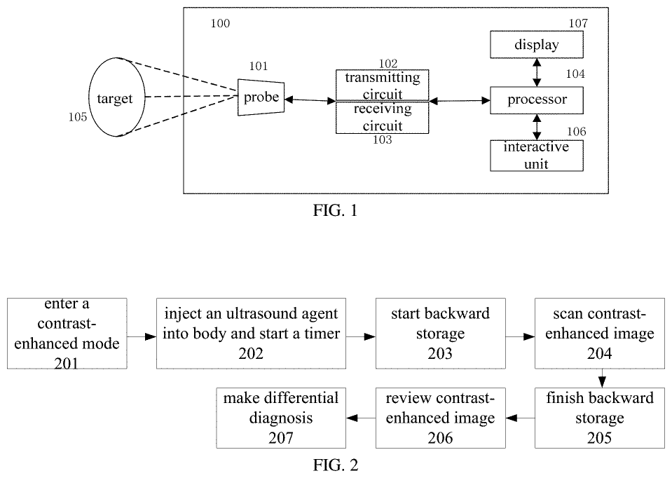

FIG. 1 shows a configuration of a CEUS imaging apparatus according to an embodiment of the present disclosure. As shown in FIG. 1, the CEUS apparatus 100 may include a probe 101, a transmitting circuit 102 for exciting the probe 101 to transmit ultrasound waves to a target 105, a receiving circuit 103 for controlling the probe 101 to receive ultrasound echo signals returned from the target 105, and a processor 104.

Various types of probes 101 may be employed, such as, but not limited to, at least one of an ultrasonic volume probe, a planar array probe, and a conventional ultrasonic array probe (such as a linear array probe, a convex array probe, and the like). CEUS image data may refer to image data generated by a CEUS apparatus under an imaging mode. The CEUS imaging apparatus 100 can be operated in at least two imaging modes including a first imaging mode and a second imaging mode, wherein the imaging velocity under the first imaging mode is faster than the imaging velocity under the second imaging mode. For example, the first imaging mode may be a high-frame-rate/high-volume-rate mode in 3D/4D CEUS imaging, so that a complete perfusion process of microbubbles at small sized lesions can be captured, while the second imaging mode may be a conventional-frame-rate/conventional-volume-rate mode in 3D/4D CEUS imaging. In this respect, doctors can freely choose one of them upon observation needs.

As shown in FIG. 1, the CEUS apparatus 100 may also include (more than one) interactive unit 106, which may be configured to perform various interactive operations by a user. The interactive unit may include, for example, but not limited to, a trackball, a knob, a touchscreen button, a gesture-sensing widget and the like. The CEUS imaging apparatus 100 may also include a display 107, which may be configured, under the control of the processor 104, to present one or more image frames to be viewed and browsed by the user, as well as to present various interfaces in the process of performing the method for viewing CEUS images according to the present disclosure to prompt the user to perform corresponding interactions. In some embodiments, the display 107 may adopt LEDs, OLEDs, etc., which will not be repeated here.

In some embodiments, the processor 104 may be a processing device including more than one general-purpose processing device, such as a microprocessor, a central processing unit (CPU), a graphics processing unit (GPU), and the like. More specifically, the processor may be a complex instruction set computing (CISC) microprocessor, a reduced instruction set computing (RISC) microprocessor, a very long instruction word (VLIW) microprocessor, a processor running other instruction sets, or a processor that runs a combination of instruction sets. The processor may also be one or more special-purpose processing devices, such as an application specific integrated circuit (ASIC), a field programmable gate array (FPGA), a digital signal processor (DSP), a system on a chip (SoC), or the like. The processor 104 may be configured to perform a method for viewing a CEUS image according to various embodiments of the present disclosure.

Please note that the present disclosure mainly takes CEUS images and CEUS apparatus as examples to describe the viewing method, but is not limited thereto, and various methods described accordingly can be flexibly applicable to various dynamic images and dynamic imaging devices that need to review and browse in a large amount of image data and find target image data, including but not limited to ordinary ultrasound images, laser ultrasound images, echocardiography, optical coherence tomography (OCT) images, and the like.

FIG. 2 shows an exemplary flowchart of a CEUS examination according to an embodiment of the present disclosure. As shown in FIG. 2, after determining by a doctor the lesion or part of a patient to be observed i.e. an observation target, the CEUS mode of the apparatus may be entered (step 201), an appropriate amount of contrast agent microbubbles may be injected into the patient and a timer of the apparatus may be started (step 202), and backward storage may be enabled simultaneously (step 203). The ultrasound probe may be used by the doctor on the observation target to scan contrast-enhanced images (step 204) to store CEUS image data in the scan in the CEUS apparatus. After the scanning of the contrast-enhanced image is completed, the backward storage may be ended (step 205). The doctor may then open the stored data to review the contrast-enhanced images (step 206) and finally make a differential diagnosis (step 207). There may be generally two browsing modes to review the contrast-enhanced images. One is to browse manually, that is, the interactive unit including a trackball, a knob or a touch-screen key on the device panel of the apparatus may be used by the user to browse in a set manual browsing step length. The other is to browse automatically, that is, the stored dynamic data may be played automatically in a set automatic browsing step length after choosing the “play” function of the apparatus by the user, just like watching a movie. During the review of the contrast-enhanced images, the two browsing modes may be switched at any time.

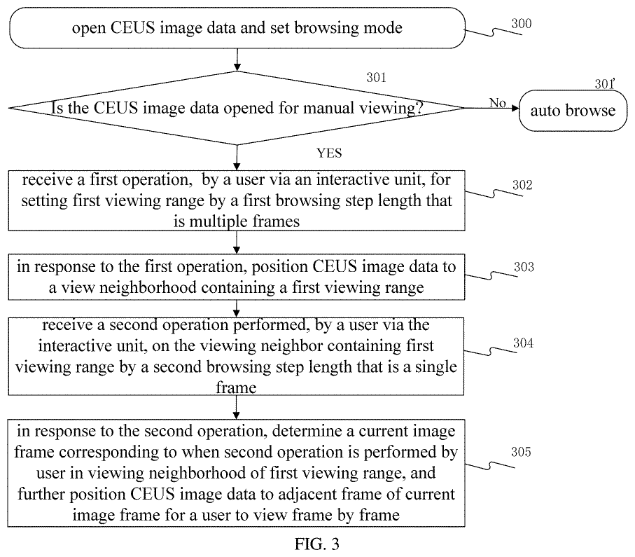

FIG. 3 shows a flowchart of Example 1 of a method for viewing a CEUS image according to an embodiment of the present disclosure. As shown in FIG. 3, the viewing method may include two stages carried out in sequence, i.e. a rough positioning stage with a first browsing step length for multiple frames and a fine positioning stage by a second browsing step length that is a single frame. As described above in conjunction with FIG. 1 the CEUS apparatus can provide a variety of imaging modes, such as but not limited to 3D imaging mode, 4D imaging mode, etc., and the first browsing step length can be set correspondingly for various imaging modes, that is, the first browsing step length can be different as the imaging mode changes. Specifically, the range of the first browsing step length corresponding to the imaging mode may be determined based on the imaging velocity corresponding to the imaging mode, so that the range of the first browsing step length corresponding to the imaging mode is positively correlated with the imaging velocity corresponding to the imaging mode. That is to say, for example, when the imaging velocity under the 3D imaging mode is faster than that under the 4D imaging mode, the setting range of the first browsing step length corresponding to the 3D imaging mode is also larger than the setting range of the first browsing step length corresponding to the 4D imaging mode. As such, for an imaging mode with a slower imaging velocity and a larger amount of information presented, a smaller first browsing step length may be automatically given to the user for reviewing and browsing in the rough positioning stage to meet the needs of performing more “dense” rough positioning for viewing and analyzing, thereby performing rough positioning more accurately and avoiding missing image frames that reflect important information. Further, a velocity ratio is positively correlated with a step-length ratio, wherein the velocity ratio may refer to a ratio of the imaging velocity of the first imaging mode to the imaging velocity of the second imaging mode, and the step-length ratio is a ratio of the first browsing step length corresponding to the first imaging mode to the first browsing step length corresponding to the second imaging mode.

As shown in FIG. 2, the stored CEUS image data is first opened, and the browsing modes including manual browsing and automatic browsing as described above is set by the user (step 300). In step 301, it may be determined whether the CEUS image data to be viewed is opened for manual viewing. The determination in step 301 actually includes two conditions: the CEUS image data to be viewed is opened, and the manual browsing mode is set (selected), thus entering the process of manual browsing. If the CEUS image data to be viewed is opened but the manual browsing mode is not selected (i.e. the determination in step 301 is NO), the automatic browsing mode is entered.

When the CEUS image data to be viewed is opened for manual viewing (the determination in step 301 is YES), a first operation of setting a first viewing range by the user via the interactive unit by the first browsing step length that is multiple frames is received (step 302). Specifically, the setting of the first viewing range may be implemented by, for example, rolling the trackball, rotating the knob, entering a numerical range (such as a time range, a frame serial number range) in an input box on the interface, and so on.

In step 303, in response to the first operation, the CEUS image data may be positioned to a viewing neighborhood containing the first viewing range. In this way, the user can quickly browse and locate the focus first viewing range by the first browsing step length for multiple frames, and a target image data to be finally located is within the first viewing range, thereby realizing efficient rough positioning. Therefore, the screening and verification of contrast-enhanced image data by users are based on rough positioning without inefficient viewing in a large amount of data.

In this specification, the term “viewing neighborhood containing the first viewing range” is intended to cover at least the viewing range of the first viewing range (which may be the first viewing range itself), and may also encompass a viewing range that is further adjacently extended with respect to the first viewing range. The CEUS image data can be positioned to the extended viewing range rather than just the first viewing range, so that an appropriate margin may be provided for, when the user performs the first operation via the interactive unit, the inertia of the operation (e.g. the roll inertia of the trackball), a deviation caused by the manual operation of the user, the judgment deviation of the user, etc., thus the target image data to be finally located may actually be located within the viewing range so as to avoid missing the target image data due to rough positioning induced by these deviations. In some embodiments, the viewing neighborhood containing the first viewing range may be a viewing range from a start time of the first viewing range or earlier to an end time of the first viewing range or later. In some embodiments, a start time of the viewing neighborhood is ahead of the starting time of the first viewing range by a first threshold, and an end time of the viewing neighborhood is behind the end time of the first viewing range by a second threshold; wherein the first and second thresholds may be predefined and/or adjustable by the user. As such, the first and second thresholds may be appropriately predefined according to the imaging mode or operational attributes of the interactive unit. Further, in addition to considering the imaging mode or the operational attributes of the interactive unit, the first and second thresholds may be adjustable by the user according to the user's own operation habits to set an appropriate deviation margin.

In step 304, a second operation of being performed on the viewing neighborhood containing the first viewing range by the user via the interactive unit by the second browsing step length that is a single frame may be received. In step 305, in response to the second operation, a current image frame corresponding to the user performing the second operation may be determined in the viewing neighborhood containing the first viewing range, and the CEUS image data may be further positioned to an adjacent frame of the current image frame for the user to view frame by frame. In the process of observing microbubble perfusion of a blood-rich and small-sized object, it is possible to miss key information about contrast-enhanced image is one frame is missed; in this respect, by providing frame-by-frame viewing after rough positioning, users can avoid missing the key information about contrast-enhanced image due to the skip of image frames, which in turn enables users to make more accurate diagnoses.

Using such a sequential execution process of rough positioning and fine positioning can not only significantly reduce the workload of users and notably improve the browsing efficiency, but also ensure that the user does not miss key information during the microbubble perfusion.

In some embodiments, various interactive units may be employed. In some embodiments, the interactive units adopted in the first operation may include a trackball, the first operation may be a rolling operation of the trackball by the user; and the interactive unit adopted in the second operation may include a knob, the second operation may be a rotating operation of the knob by the user. On the CEUS apparatus, the operations of the trackball and the knob are familiar and user-friendly, and are compatible with the settings of trackballs and knobs of the existing CEUS apparatus, thereby reducing manufacturing costs. Further, by performing the second operation by the second browsing step length that is a single frame via the knob, a more accurate step can be performed. Specifically, for example, the rotating operation of the knob may also be discrete, such as turning the knob for one step representing going to a next single frame, so that the user can better control the accuracy of the second operation, thereby avoiding missing the key information about contrast-enhanced image due to operation deviation.

In some embodiments, a single trackball may be served both as an interactive unit utilized by the first operation and the second operation. The first operation and the second operation are the rolling operation of the single trackball by the user to make the browsing step length for multiple frames and a single frame respectively. For example, the first operation may be performed by rolling the trackball quickly, and the second operation may be performed by rolling the trackball slowly. In this way, the configuration of the interactive unit can be made more compact, avoiding the distraction of the user's attention caused by switching among different types of interactive units, and improving the operation efficiency of the user.

In some embodiments, each interactive unit may be implemented via a touch button on the touch screen. In this way, there is no need to rely on hardware configuration to implement each interactive unit, and the first operation and the second operation can be performed by the user by touching and clicking each touch button on the touch screen, so that the user only needs to pay attention to the touch screen, improving the operation fluency. In some embodiments, each interactive unit may also be implemented via a gesture sensing component, thereby freeing the user's hands and avoiding cross infection.

In some embodiments, the interactive unit utilized in the first operation may be a time input unit on the touch screen, and the first operation may be setting the definition of time information corresponding to the first viewing range by the user via the time input unit.

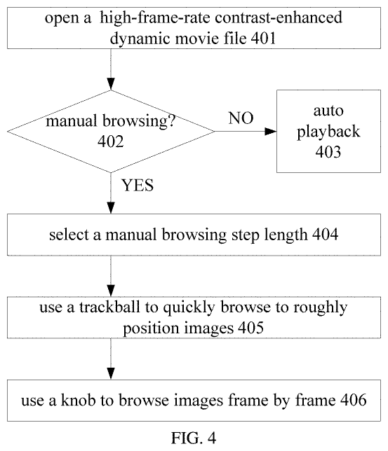

FIG. 4 shows a flowchart of Example 2 of a method of viewing a CEUS image according to an embodiment of the present disclosure, wherein a trackball is employed for the first operation and a knob is employed for the second operation. As shown in FIG. 4, in step 401, a high frame rate contrast-enhanced dynamic movie file may be opened. In step 402, it is determined whether to enter the manual browsing mode; and when the determination is NO, an automatic playback mode may be entered (step 403).

In the case of entering the manual browsing mode (i.e. the determination of step 402 is YES), the manual browsing step length may be selected (or set) by the user (step 404). After the manual browsing step length is selected, the trackball may be used to quickly browse and roughly position the images (step 405). After the rough positioning of the images, by using the knob to browse the images frame by frame by the user within the viewing range of the rough positioning (step 406), target images can be screened accurately with avoidance of omission.

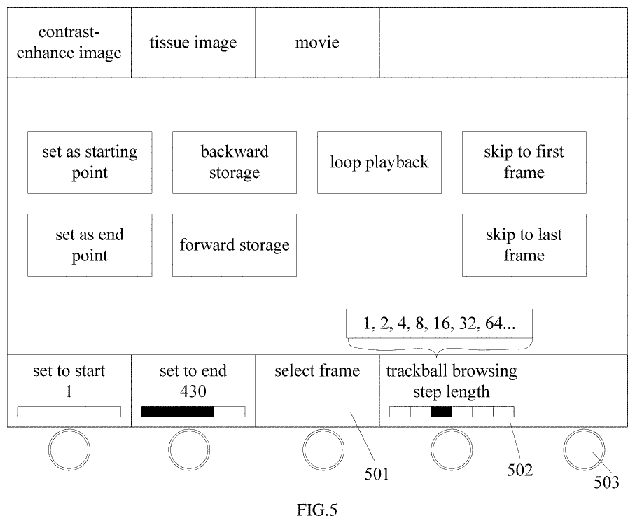

FIG. 5 shows an interface for manually viewing and browsing CEUS images according to an embodiment of the present disclosure. After opening the contrast-enhanced dynamic image data to be browsed, the contrast-enhanced images can be browsed sequentially in multiple frames (rough positioning) or frame by frame (fine positioning) through the trackball (not shown) or the knob 503 on the panel of the CEUS apparatus.

An image display area may be updated to show adjacent frames of the current image by slowly moving the trackball or turning the knob 503. In the manual browsing mode, the step length of the knob or the trackball for scroll browsing may be one frame of contrast-enhanced image (for 3D mode, and the step length may be one volume of contrast-enhanced images for 4D mode). If the doctor needs to browse the contrast-enhanced images quickly, it can only be achieved by quickly rolling the trackball or rapidly turning the knob 503; in this way, the system may update the image of a corresponding frame according to the user's moving speed, the faster the update. As shown in FIG. 5, the contrast-enhanced image can be selected frame by frame for reviewing and browsing by rotating the knob 503 under a control of “select frame” 501.

In some embodiments, the first browsing step length for rough positioning may be selected by the user from a plurality of step-length levels. As shown in FIG. 5, the forward step length corresponding to rolling the trackball for once, such as 1, 2, 4, 8, 16, 32, 64, etc., may be chosen by using the knob 503 under a control of “browsing step length of trackball” 502. For example, if the selected step length is 32, then with moving the trackball once, the system may choose the image at the 32^{nd} frame of the current frame in a corresponding moving direction for display. When manually browsing high frame rate contrast-enhanced data, the doctor may start by selecting a high level to find an approximate location of the frame of interest more quickly. Then, through the knob 503 under the control of “select frame” 501, the contrast-enhanced images are browsed and played back frame by frame in the vicinity of the location.

In some embodiments, the sequential rough positioning and fine positioning of the present disclosure is not limited to being implemented in the manual browsing mode (also referred to as a manual viewing mode), but may also be realized in the automatic browsing mode (also referred to as an automatic viewing mode), or be implemented in in combination with the automatic browsing mode and the manual browsing mode (i.e. semi-automatic browsing mode).

Specifically, take rough positioning as an example for description. The rough positioning in manual viewing mode may be implemented by the processor performing the following steps. A prompt may be presented to the user to select the manual viewing mode. In response to a fourth operation in which the user selects the manual viewing mode, an interactive interface corresponding to the manual viewing mode may be presented to the user. Based on the interactive interface, the first operation of the user setting the first viewing range by the first browsing step length of multiple frames may be received.

For rough positioning or fine positioning, the browsing step length can be set by selecting the playback speed in the automatic viewing mode. Specifically, the user can be prompted to select the automatic viewing mode. In response to a fifth operation in which the user selects the automatic viewing mode, the interactive interface corresponding to the automatic viewing mode may be presented to the user. On the interactive interface corresponding to the automatic viewing mode, a selectable item involving playback speed may be presented on the interactive interface corresponding to the automatic viewing mode for the user to select.

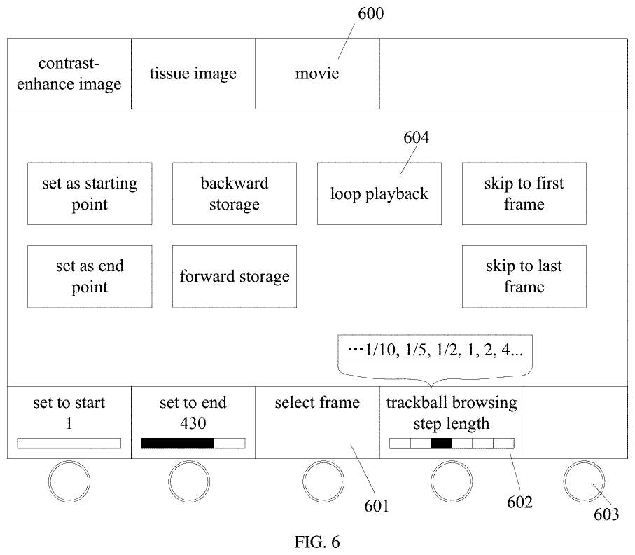

For example, rough positioning can be implemented in automatic browsing mode, followed by fine positioning in manual browsing mode. As shown in FIG. 6, after opening the dynamic contrast-enhanced image data to be browsed, the automatic playing (automatic playback) of the dynamic contrast-enhanced image may be enabled by triggering a touch screen button “movie” 600 of the CEUS apparatus, and the playback speed can be selected. Playing with 1× speed may be equivalent to playing with the frame rate at which the dynamic contrast-enhanced data was stored. When the playback speed is greater than 1× and less than 1×, the effect observed by the user is similar to “fast-motion playback” and “slow-motion playback”. The setting of the playback speed may be selected by the knob 603 or a button. For example, by pressing the knob 603 under a control of “automatic playback” 602, the user may choose whether to start automatic playback; and by rotating the knob 603, the user may choose the playback speed. For example, the playback speed of a movie in this example may be selected from 1/10, ⅕, ½, 1, 2, 4 . . . etc. In some embodiments, after pressing a “loop play” button 604 on the touch screen, the system may automatically skip to the start frame to continue playing every time it plays to the end frame, thus it may play repeatedly.

Rough positioning may be implemented by watching the dynamic contrast-enhanced image data played at the selected playback speed (as an example of “first browsing step”) by the user. For example, when the user thinks that the viewing range for rough positioning has been found, the automatic playback may be ended by pressing the knob 603 under the control of “automatic playback” or by pressing the knob 603 under the control of “select frame” 601, and the image frames in the viewing range corresponding to rough positioning may be displayed on the screen for the users to view frame by frame. Then, one or several target image frames containing key information can be found by the user by rotating the knob 603 under the control of “select frame” 601 with one single frame per one step length, thus implementing fine positioning.

In some embodiments, rough positioning may also be implemented in manual browsing mode, followed by fine positioning in automatic browsing mode. Specifically, for the CEUS image data, that is, the image data generated by the CEUS apparatus under the imaging mode, the imaging mode may include a first imaging mode and a second imaging mode, wherein the imaging velocity under the first imaging mode is faster than the imaging velocity under the second imaging mode, and the imaging velocity is positively correlated by the first browsing step length. The viewing method may include, where the CEUS image data to be viewed is opened for manual viewing, receiving the first operation of setting the first viewing range by the user via the interactive unit by the first browsing step length that is a plurality of frames. In response to the first operation, the CEUS image data may be positioned to a viewing neighborhood containing the first viewing range. Next, a fifth operation by the user for setting an automatic viewing mode may be received; and in response to the fifth operation, the CEUS image data may begin to play automatically in a single-frame manner in the viewing neighborhood containing the first viewing range. The playback speed of the automatic playback is lower than the imaging velocity of the second imaging mode, so that the desired information can be smoothly presented to the user during the automatic playback.

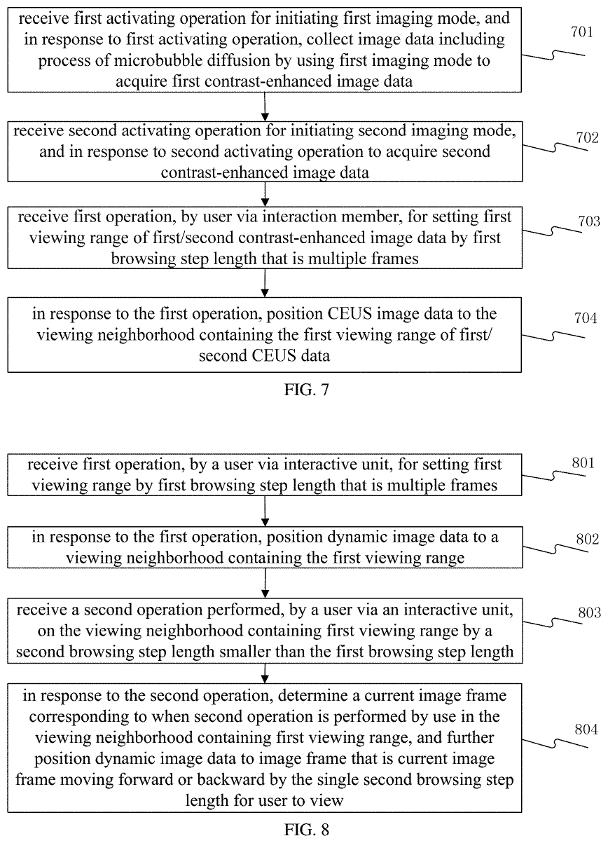

FIG. 7 shows a flowchart of Example 3 of a method for viewing a CEUS image according to an embodiment of the present disclosure. The viewing method is described in the case of at least two imaging modes including the first imaging mode and the second imaging mode. Accordingly, the CEUS image data may include first contrast-enhanced image data acquired under the first imaging mode and second contrast-enhanced image data under the second imaging mode. For example, the first imaging mode may be a high frame rate/high volume rate imaging mode to enable acquisition of image data during microbubble wash-in, and the second imaging mode may be a conventional frame rate/conventional volume rate imaging mode.

As shown in FIG. 7, in step 701, a first activating operation for initiating the first imaging mode may be received, and in response to the first activating operation, the image data including the process of microbubble wash-in can be collected using the first imaging mode to obtain the first contrast-enhanced image data.

In step 702, a second activating operation for initiating the second imaging mode may be received, and the second contrast-enhanced image data may be obtained in response to the second activating operation.

In step 703, the first operation of setting the first viewing range of the first contrast-enhanced image data/the second contrast-enhanced image data by the user via the interactive unit by the first browsing step length that is multiple frames may be received.

At step 704, in response to the first operation, the CEUS image data may be positioned to the viewing neighborhood containing the first viewing range of the first CEUS data/the second CEUS data.

In this way, it is possible to switch between the first imaging mode and the second imaging mode as required to obtain corresponding contrast-enhanced image data under a desired imaging mode, and perform rough positioning for the contrast-enhanced image data. The details of the implementation of rough positioning and fine positioning according to various embodiments of the present disclosure may be combined here as needed, which will not be repeated here. Correspondingly, the selection range of the first browsing step length may be different according to the imaging mode. For example, the range of the first browsing step length corresponding to the imaging mode may be positively correlated with the imaging velocity corresponding to the imaging mode.

Though the aforesaid viewing method is described by taking CEUS data as an example, it should be noted that the viewing method can be extended to various dynamic images and dynamic imaging apparatus that need to review and browse a large amount of image data and find the target image data, such as but not limited to ordinary Ultrasound images, laser ultrasound images, echocardiograms, optical coherence tomography (OCT) images, and more.

FIG. 8 shows a flowchart of Example 4 of a method for viewing a dynamic image according to an embodiment of the present disclosure, which is performed when dynamic image data including multiple image frames to be viewed is opened for manual viewing. When the dynamic image data including multiple image frames to be viewed is opened for manual viewing, the first operation, by the user via the interactive unit, for setting the first viewing range by the first browsing step length that is multiple frames may be received (step 801).

In step 802, in response to the first operation, the dynamic image data may be positioned to the viewing neighborhood containing the first viewing range.

In step 803, the second operation of being performed, by the user via the interactive unit, on the viewing neighborhood containing the first viewing range by the second browsing step length may be received, wherein the second browsing step length is smaller than the first browsing step length. For dynamic data of different modes, the first browsing step length and the second browsing step length may be set appropriately. For example, the first browsing step length may be set according to the imaging velocity of a corresponding mode, so as to ensure that the image information can be presented smoothly during rough positioning. For another example, the second browsing step length may be set according to the user's observation requirements; for instance, in the CEUS image data, the second browsing step length may be set as a single frame to capture the process of microbubble perfusion, but it may also be set as several image frames and the like in other modes or for other observation needs. The second browsing step length may be set upon specific needs, or be adjusted independently by the user to meet the needs of the user in different application scenarios.

In step 804, in response to the second operation, a current image frame corresponding to the second operation performed by the user may be determined in the viewing neighborhood containing the first viewing range, and the dynamic image data may be further positioned to the single image frame that corresponds to the current image frame going forward or backward the second browsing step length for the user to view.

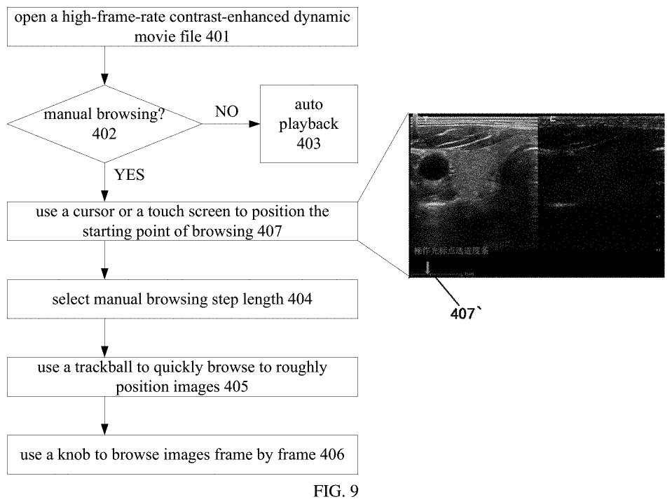

FIG. 9 shows a flowchart of Example 5 of a method for viewing a CEUS image according to an embodiment of the present disclosure. Steps 401 to 406 in FIG. 9 are similar to steps 401 to 406 in FIG. 4, and details thereof are not repeated here. The difference therebetween is that, before receiving the first operation, by the user via the interactive unit, for setting the first viewing range by the first browsing step length that is multiple frames, that is to say, before the manual browsing step length used for rough positioning is selected by the user (404), the starting position of browsing is located by using a cursor or touch screen (step 407). As shown in FIG. 9, the cursor may be displayed when a high frame rate contrast-enhanced file is opened by the user, and a progress bar 407′ may be clicked by operating the cursor by the user. A specific position of the progress bar may be clicked directly by the user, and a slider may be skipped to the position.

Correspondingly, the processor can detect the user's click operation on the progress bar to locate the CEUS image data to a third viewing range corresponding to the clicked position on the progress bar, and present the corresponding image of the third viewing range. The corresponding image may be displayed on a main screen, as shown in FIG. 9.

After presenting the image corresponding to the third viewing range, the processor may receive the first operation, by the user via the interactive unit, for setting the first viewing range by the first browsing step length that is multiple frames, thereby completing rough positioning. Then, the user can further use the trackball or knob to perform fine positioning, such as but not limited to browsing the images frame by frame.

In addition to the progress bar, rough positioning may also be performed on high frame rate contrast-enhanced images by various ways.

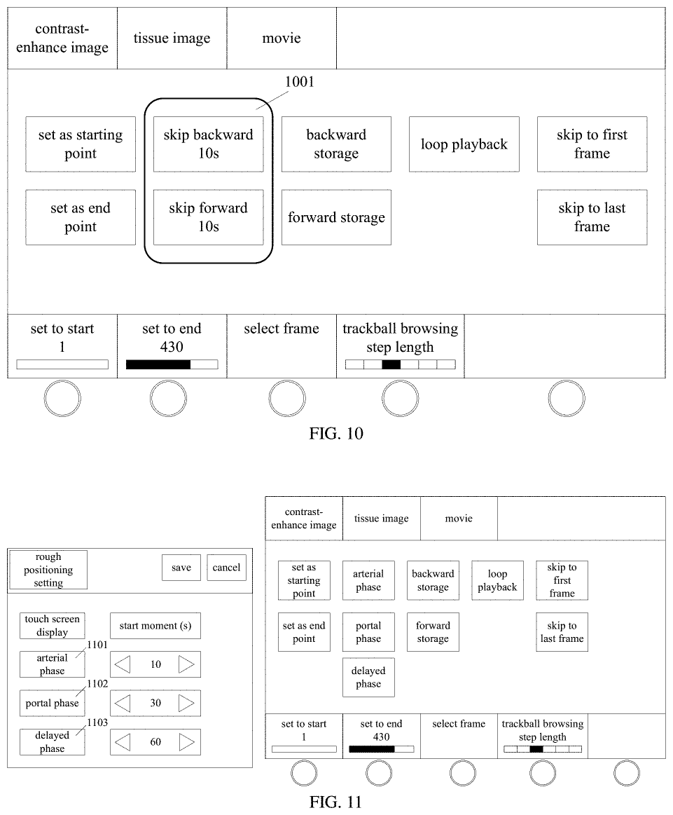

In some embodiments, the interactive unit utilized by the first operation may be a time input unit (e.g., an input box) on the touch screen, and the first operation may be setting via the time input unit by the user to define time information corresponding to the first viewing range. As shown in FIG. 10, controls of “skip backward 10 s” and “skip forward 10 s” 1001 on the touch screen represent directly skipping to the time after or before 10 seconds based on the time corresponding to the current image, and displaying the image corresponding to the skipped time simultaneously on the main screen. Then, the user can further use the trackball or the knob “select frame” to browse, for example, first browsing quickly by the first browsing step length that is multiple frames and then browsing finely by the second browsing step length that is a single frame; alternatively, the user may also directly choose a surrounding neighborhood at the time after or before 10 seconds of current moment as the first viewing range for rough positioning, and then only browse finely by the second browsing step length that is a single frame. The ten seconds here is just an example, the system can support an actual skip range that is defined independently by the user in the system initialization.

In some embodiments, the first viewing range is set in association with each phase of the microbubble wash-in and wash-out process of the contrast agent injected in the target object, and the interactive unit used in the first operation may include buttons on the touch screen that are set to correspond to each phase; accordingly, the first operation may be an operation of pressing each button.

For example, for a specific application such as CEUS in liver imaging, buttons, including an “arterial phase” button 1101, a “portal phase” button 1102 and a “delayed phase” button 1103 which are corresponded to 10 seconds, 30 seconds and 60 seconds respectively, may be set, as shown in FIG. 11. The left side is an editing interface, and the right side is a touch screen interface after editing. When the “arterial phase” button 1101 is clicked by the user, the image shown on the main screen may be directly skipped to the moment of 10 seconds forward/backward and updated to display. Then, fine browsing can be further performed by using the trackball or the “select frame” knob by the user. Specifically, the system can be given better flexibility for rough positioning, and the names and specific corresponding times of buttons on the touch screen can be edited and set by the user. By setting the first viewing range of rough positioning in association with each medical application, the comprehensibility of time setting for doctors can be improved, thus enhancing diagnostic efficiency.

In some embodiments, an apparatus for viewing a CEUS image is provided, and the CEUS image data is generated by the CEUS apparatus under the imaging mode. Note that the viewing apparatus may be integrated into the CEUS apparatus, or be realized as other means in communication with the CEUS apparatus to receive the CEUS image data therefrom. In some embodiments, the viewing apparatus may be located at other terminals, such as, but not limited to, a user's portable terminal, a terminal of an image station, a server in the cloud, and the like. The imaging mode may include a first imaging mode and a second imaging mode. The imaging velocity under the first imaging mode is faster than that under the second imaging mode. The range of the first browsing step length corresponding to the imaging mode is determined based on the imaging velocity corresponding to the imaging mode, and the range of the first browsing step length corresponding to the imaging mode is positively correlated with the imaging velocity corresponding to the imaging mode.

Specifically, the viewing apparatus may include an interactive unit, a processor, and a display. The implementations of the interactive unit, the processor and the display according to the various embodiments of the present disclosure may be combined herein, which will not be repeated here.

In some embodiments, the interactive unit may be configured as: performing the first operation by the user for setting the first viewing range by the first browsing step length that is multiple frames; and performing the second operation by the user on the viewing neighborhood containing the first viewing range by the second browsing step length that is a single frame. The processor may be configured to perform the method for viewing the CEUS image and each step thereof according to various embodiments of the present disclosure. The display may be configured to present, under the control of the processor, a corresponding image or interface of the positioned viewing range.

In some embodiments, the interactive unit may be configured as: performing the first operation by the user for setting the first viewing range by the first browsing step length that is a plurality of frames; and performing the fifth operation by the user for setting the automatic viewing mode. The processor may be configured to: receive the first operation, by the user via the interactive unit, for setting the first viewing range by the first browsing step length that is multiple frames; in response to the first operation, position the CEUS image data to the viewing neighborhood containing the first viewing range; receive the fifth operation by the user for setting the automatic viewing mode; and in response to the fifth operation, enable to automatically play the CEUS image data within the viewing neighborhood containing the first viewing range in a single-frame manner, wherein the playback speed of the automatic playback is lower than the imaging velocity under the second imaging mode. The display may be configured to present, under control of the processor, an image corresponding to the located viewing range.

In some embodiments, an apparatus for viewing a dynamic image may be provided. The viewing apparatus may include an interactive unit, a processor and a display. The implementations of the interactive unit, the processor, and the display according to the various embodiments of the present disclosure may be combined herein, which will not be repeated here.

The interactive unit may be configured as: performing the first operation by the user for setting the first viewing range with the first browsing step length that is multiple frames; and performing the second operation by the user on the viewing neighborhood containing the first viewing range by the second browsing step length that is smaller than the first browsing step length.

The processor may be configured to perform the following steps when the dynamic image data including multiple image frames to be viewed is opened for manual viewing. The first operation performed by the user may be detected. In response to the first operation, the dynamic image data is positioned to the viewing neighborhood containing the first viewing range. The second operation performed by the user may be detected. In response to the second operation, the current image frame corresponding to the user performing the second operation is determined in the viewing neighborhood containing the first viewing range, and the dynamic image data is further positioned to the image frame that is the current image frame moving forward or backward by one single second browsing step length.

The display may be configured to present a corresponding image of the positioned viewing range.

The present disclosure also provides a computer-readable storage medium on which computer-executable instructions are stored, which, when executed by a processor, realizes part or all of the processing of the method for viewing CEUS images or dynamic images according to various embodiments of the present disclosure. Some or all of the processing can be implemented as a computer program. The above-described program can be stored in various types of non transitory computer-readable media and can be provided to a computer. Non-transitory computer-readable media include various types of tangible storage media. Examples of non-transitory computer-readable media include magnetic recording media (such as floppy disks, magnetic tapes, and hard disk drives), magneto-optical recording media (such as magneto-optical disks), read only memory (CD-ROM), CD-R, CD-R/W, semiconductor memory (such as mask ROM, programmable ROM (PROM), erasable Prom (EPROM), flash ROM, and random access memory (RAM)). The program may be provided to the computer via various types of transitory computer-readable media. Examples of transitory computer-readable media include electrical signals, optical signals, and electromagnetic waves. The transitory computer-readable media can provide the program to the computer via wired communication paths (such as electric wires and optical fibers) or wireless communication paths.

The present invention is not limited to the above-described embodiments and can be modified as necessary without departing from the scope of the present invention.

1. A method for viewing a contrast-enhanced ultrasound image, wherein data of the contrast-enhanced ultrasound image is generated under an imaging mode by a contrast-enhanced ultrasound apparatus, the imaging mode comprises a first imaging mode and a second imaging mode, an imaging velocity under the first imaging mode is faster than that under the second imaging mode, a range of a first browsing step length corresponding to the imaging mode is determined upon the imaging velocity corresponding to the imaging mode, the range of the first browsing step length corresponding to the imaging mode is positively correlated with the imaging velocity corresponding to the imaging mode, and the method comprises: by means of a processor, when the contrast-enhanced ultrasound image data to be viewed is opened for manual viewing:
receiving a first operation, by a user via an interactive unit, for setting a first viewing range by the first browsing step length that is multiple frames;
positioning the contrast-enhanced ultrasound image data to a viewing neighborhood containing the first viewing range in response to the first operation;
receiving a second operation performed, by the user via the interactive unit, on the viewing neighborhood containing the first viewing range by a second browsing step length that is a single frame; and
in response to the second operation, determining a current image frame corresponding to when the second operation is performed by the user in the viewing neighborhood containing the first viewing range, and further positioning the contrast-enhanced ultrasound image data to an adjacent frame of the current image frame for the user to view frame by frame.

2. (canceled)

3. The method for viewing according to claim 2, wherein a start time of the viewing neighborhood is ahead of the starting time of the first viewing range by a first threshold, an end time of the viewing neighborhood is behind the end time of the first viewing range by a second threshold, and the first threshold and the second threshold are predefined and/or adjustable by the user.

4. The method for viewing according to claim 1, wherein the interactive unit used in the first operation comprises a trackball, accordingly the first operation is a rolling operation of the trackball by the user; and the interactive unit used in the second operation comprises a knob, accordingly the second operation is a rotating operation of the knob by the user.

5. The method for viewing according to claim 1, wherein a single trackball is served as the interactive unit used in both the first operation and the second operation, and the first operation and the second operation are the rolling operation of the single trackball by the user to make the browsing step length as multiple frames and a single frame respectively.

6. The method for viewing according to claim 1, wherein each interactive unit is implemented via a touch button on a touch screen, and/or via a gesture sensing component.

7. The method for viewing according to claim 1, wherein the first browsing step length is selected by the user from a plurality of step-length levels.

8. The method for viewing according to claim 1, wherein the first viewing range is set in association with each phase of microbubble wash-in and wash-out of a contrast agent injected in a target object, the interactive unit used in the first operation comprises buttons on the touch screen that are respectively set to correspond to each phase, and the first operation comprises an operation of pressing each button.

9. The method for viewing according to claim 1, wherein the contrast-enhanced ultrasound image data comprises a first contrast-enhanced imaging data and a second contrast-enhanced imaging data, and the method for viewing further comprises:
receiving a first activating operation for initiating the first imaging mode, and in response to the first activating operation, collecting imaging data including a process of microbubble wash-in by using the first imaging mode to acquire the first contrast-enhanced image data;
receiving a second activating operation for initiating the second imaging mode, and obtaining the second contrast-enhanced image data in response to the second activating operation;
receiving the first operation of setting the first viewing range of the first/second contrast-enhanced image data by the user via the interactive unit by the first browsing step length that is multiple frames; and
in response to the first operation, positioning the contrast-enhanced ultrasound image data to the viewing neighborhood containing the first viewing range of the first/second contrast-enhanced ultrasound image data.

10. The method for viewing according to claim 1, wherein the interactive unit used in the first operation comprises a time input unit on the touch screen, and the first operation comprises setting a definition of time information corresponding to the first viewing range by the user via the time input unit.

11. The method for viewing according to claim 1, further comprising: by means of the processor, before receiving the first operation of setting the first viewing range by the user via the interactive unit by the first browsing step length that is multiple frames:
detecting a click operation by the user on a progress bar to position the contrast-enhanced ultrasound imaging data to a third viewing range corresponding to a clicked point on the progress bar, and presenting an image corresponding to the third viewing range; and
after presenting the image corresponding to the third viewing range, performing, by the user via the interactive unit, the step of receiving the first operation of setting the first viewing range by the first browsing step length that is multiple frames.

12-13. (canceled)

14. The method for viewing according to claim 1, wherein a velocity ratio is positively correlated with a step-length ratio, the velocity ratio referring to a ratio of the imaging velocity of the first imaging mode to the imaging velocity of the second imaging mode, and the step-length ratio is a ratio of the first browsing step length corresponding to the first imaging mode to the first browsing step length corresponding to the second imaging mode.

15. (canceled)

16. A method for viewing a dynamic image, for viewing dynamic image data comprising multiple image frames, comprising: by means of a processor, when the dynamic image data comprising multiple image frames to be viewed is opened for manual viewing:
receiving a first operation, by a user via an interactive unit, for setting a first viewing range by a first browsing step length that is multiple frames;
in response to the first operation, positioning the dynamic image data to a viewing neighborhood containing the first viewing range;
receiving a second operation performed, by the user via the interaction unit, on the viewing neighborhood containing the first viewing range by a second browsing step length, the second browsing step length being smaller than the first browsing step length; and
in response to the second operation, determining a current image frame corresponding to when the second operation is performed by the user in the viewing neighborhood containing the first viewing range, and further positioning the dynamic image data to an image frame that is the current image frame moving forward or backward by one single second browsing step length for the user to view frame by frame.

17. The method for viewing according to claim 16, wherein the second browsing step length is a step length that is a single frame for the user to view frame by frame.

18. (canceled)

19. The method for viewing according to claim 18, wherein a start time of the viewing neighborhood is ahead of the starting time of the first viewing range by a first threshold, an end time of the viewing neighborhood is behind the end time of the first viewing range by a second threshold, and the first threshold and the second threshold are predefined and/or adjustable by the user.

20. The method for viewing according to claim 16, wherein the interactive unit used in the first operation comprises a trackball, accordingly the first operation is a rolling operation of the trackball by the user; and the interactive unit used in the second operation comprises a knob, accordingly the second operation is a rotating operation of the knob by the user.

21. The method for viewing according to claim 16, wherein a single trackball is served as the interactive unit used in both the first operation and the second operation, and the first operation and the second operation are the rolling operation of the single trackball by the user to make the browsing step length as multiple frames and a single frame respectively.

22-24. (canceled)

25. The method for viewing according to claim 16, wherein the dynamic image data comprises dynamic image data acquired by contrast-enhanced ultrasound imaging, the first viewing range is set in association with each phase of microbubble wash-in and wash-out of a contrast agent injected in a target object, the interactive unit used in the first operation comprises buttons on the touch screen that are respectively set to correspond to each phase, and the first operation comprises an operation of pressing each button.

26. The method for viewing according to claim 16, wherein the interactive unit used in the first operation comprises a time input unit on the touch screen, and the first operation comprises setting a definition of time information corresponding to the first viewing range by the user via the time input unit.

27. The method for viewing according to claim 16, further comprising: by means of the processor, before receiving the first operation of setting the first viewing range by the user via the interactive unit by the first browsing step length that is multiple frames:
detecting a click operation by the user on a progress bar to position the dynamic imaging data to a third viewing range corresponding to a clicked point on the progress bar, and presenting an image corresponding to the third viewing range; and
after presenting the image corresponding to the third viewing range, performing, by the user via the interactive unit, the step of receiving the first operation of setting the first viewing range by the first browsing step length that is multiple frames.

28-29. (canceled)

30. An apparatus for viewing a contrast-enhanced ultrasound image, wherein data of the contrast-enhanced ultrasound image is generated under an imaging mode by a contrast-enhanced ultrasound apparatus, the imaging mode comprises a first imaging mode and a second imaging mode, an imaging velocity under the first imaging mode is faster than that under the second imaging mode, the imaging velocity is positively correlated with a first browsing step length, and
the apparatus for viewing comprises:
an interactive unit configured to perform a first operation by the user for setting a first viewing range by the first browsing step length that is multiple frames, and perform a second operation by the user on a viewing neighborhood containing the first viewing range by a second browsing step length that is a single frame;
a processor configured to:
detect a first operation performed by the user;
in response to the first operation, position the contrast-enhanced ultrasound image data to a viewing neighborhood containing the first viewing range;
detect a second operation performed by the user; and
in response to the second operation, determining a current image frame corresponding to when the second operation is performed by the user in the viewing neighborhood containing the first viewing range, and further positioning the contrast-enhanced ultrasound image data to an adjacent frame of the current image frame for the user to view frame by frame; and
a display configured to show an image or interface corresponding to the positioned viewing range under the control of the processor.

31-42. (canceled)
